(19) United States
(12) Patent Application Publication
(10) Pub. No.: US 2025/0099977 A1
(43) Pub. Date: Mar. 27, 2025

(54) METHOD FOR MANUFACTURING ELECTROSTATIC FILTER AND FILM FOR PATHOGEN INACTIVATION

(71) Applicant: UIF (University Industry Foundation), Yonsei University, Seoul (KR)

(72) Inventors: Sang Woo KIM, Seoul (KR); In Yong SUH, Seoul (KR); Jin Young JEON, Paju-si (KR)

(73) Assignee: UIF (University Industry Foundation), Yonsei University, Seoul (KR)

(21) Appl. No.: 18/891,114

(22) Filed: Sep. 20, 2024

(30) Foreign Application Priority Data
Sep. 22, 2023 (KR) 10-2023-0126695

(51) Int. Cl. B03C 3/28; A61L 9/22; B03C 3/62; B03C 3/64; B03C 3/70; C01B 32/186; C23C 16/01; C23C 16/26

(52) U.S. Cl. CPC B03C 3/28; A61L 9/22; B03C 3/62; B03C 3/64; B03C 3/70; C01B 32/186; C23C 16/01; C23C 16/26; A61L 2209/14; C01B 2204/02

(57) ABSTRACT

The present invention relates to an electrostatic filter and film for pathogen inactivation, and furthermore, to a method for manufacturing the electrostatic filter and film for pathogen inactivation. The present invention relates to an electrostatic filter and film for pathogen inactivation using large-area charge injection into graphene, and using the graphene having improved charge retention ability.

### CROSS-REFERENCE TO RELATED APPLICATION(S)

This application claims the benefit under 35 USC 119 (a) of Korean Patent Application No. 10-2023-0126695, filed with the Korean Intellectual Property Office on Sep. 22, 2024, the disclosure of which is incorporated herein by reference in its entirety.

### BACKGROUND

### Field

The present disclosure relates to an electrostatic filter and film for pathogen inactivation, and furthermore, to a method for manufacturing the electrostatic filter and film for pathogen inactivation. The present disclosure relates to an electrostatic filter and film for pathogen inactivation using large-area charge injection into graphene and using the graphene having improved charge retention ability.

### Description of Related Art

In recent years, numerous people have lost their lives worldwide due to airborne pathogens including influenza, pneumonia, and especially coronavirus (COVID-19), and such epidemics occur approximately every five years.

The most commonly used method for preventing airborne (droplet infection) pathogen infection is a face mask. However, face masks cannot be worn for long periods of time due to pressure drop and discomfort. Furthermore, the filter should be replaced periodically.

Another preventive method is the HEPA (High-efficiency particulate air) filter, which is mainly used to remove pathogens and particulates in air conditioning systems. Although the HEPA filter can physically block pathogens, it does not inactivate pathogens, such that there is a problem of secondary infection when replacing the filter. Furthermore, pressure drop makes it difficult for air to pass through the filter, and additional energy may be required to use the filter. The physical blocking method using the filter using electrostatic electricity as mentioned above has the problem that pathogen capture efficiency thereof decreases over time.

Therefore, research is needed to improve the pathogen capture efficiency of the filter using electrostatic electricity. In particular, the charge existing in the electrostatic filter should be maintained for pathogen control even in high-temperature and humid environments such as inside a mask and in summer. Using the charge maintenance characteristic, it is expected that the filter will not need to be replaced periodically, and not physical blocking but electrical blocking in the HEPA filter will be achieved. Furthermore, it is expected that pathogen infection may be prevented in areas where people touch a lot, such as elevators and handles, by using electrostatic films.

### SUMMARY

A purpose of the present disclosure is to provide an electrostatic filter and film to which graphene technology which maintains a large area of charges for a long time, has a high sterilization effect even in a high temperature and humidity environment, and controls pathogens is applied.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

A first aspect of the present disclosure provides an electrostatic filter and film for pathogen inactivation, comprising: an insulating layer disposed on a substrate; and a monolayer graphene layer disposed on the insulating layer, wherein a metal foil is placed on an upper surface of the graphene layer, and the metal foil is pressed against the graphene layer under a physical force so as to cause friction therebetween while a bias voltage is applied to the metal foil, such that electric charges are locally stored in an area between the graphene layer and the insulating layer under a tunneling effect.

In accordance with some embodiments of the electrostatic filter and film for pathogen inactivation, the insulating layer includes one selected from SiO₂, Al₂O₃, and stretchable/flexible polymer (PET, PU, PDMS, PI, etc.).

In accordance with some embodiments of the electrostatic filter and film for pathogen inactivation, the graphene layer has an area of 1 centimeter square or larger.

In accordance with some embodiments of the electrostatic filter and film for pathogen inactivation, the metal foil is a copper foil, wherein the physical force is applied using a weight.

In accordance with some embodiments of the electrostatic filter and film for pathogen inactivation, the charge injected into the graphene layer is stored for 72 hours at a temperature of 60° C. and a humidity of 90%.

In accordance with some embodiments of the electrostatic filter and film for pathogen inactivation, after using the electrostatic filter and film for a predefined period of time, the metal foil is placed on the upper surface of the graphene layer, and the metal foil is pressed against the graphene layer under a physical force so as to cause friction therebetween while the bias voltage is applied to the metal foil, such that new electric charges are re-stored in the area between the graphene layer and the insulating layer under a tunneling effect, and thus the electrostatic filter and film is reusable.

A second aspect of the present disclosure provides a method for manufacturing an electrostatic filter and film for pathogen inactivation, the method comprising: a step of forming an insulator layer on a substrate; a step of preparing a monolayer graphene layer and transferring the prepare monolayer graphene layer onto the insulator layer in a wet transfer manner; and a step of placing a metal foil on the graphene layer, placing a weight on the metal foil, and then applying a voltage to the metal foil to inject electrical charges into the graphene layer, such that electric charges are locally stored in an area between the graphene layer and the insulating layer under a tunneling effect.

In accordance with some embodiments of the method for manufacturing the electrostatic filter and film for pathogen inactivation, the step of preparing the monolayer graphene layer and transferring the prepare monolayer graphene layer onto the insulator layer in the wet transfer manner includes: a step of synthesizing a monolayer graphene on a copper foil using a chemical vapor deposition; a step of spin-coating PMMA (polymethyl methacrylate) on the graphene synthesized on the copper foil; a step of immersing the PMMA-coated graphene into a copper etchant to remove copper therefrom; and a step of transferring the graphene onto the insulating layer in a wet transfer manner.

In accordance with some embodiments of the method for manufacturing the electrostatic filter and film for pathogen inactivation, the insulating layer includes one selected from SiO₂, Al₂O₃, and stretchable/flexible polymer (PET, PU, PDMS, PI, etc.).

In accordance with some embodiments of the method for manufacturing the electrostatic filter and film for pathogen inactivation, the graphene layer has an area of 1 centimeter square or larger.

The electrostatic filter and film according to the present disclosure may maintain charges over a large area inside the graphene via charge injection technology, and thus may inactivate surrounding pathogens.

Effects of the present disclosure are not limited to the effects mentioned above, and other effects not mentioned will be clearly understood by those skilled in the art from the descriptions below.

### BRIEF DESCRIPTION OF DRAWINGS

FIG. 1 is a schematic diagram of an electrostatic filter for pathogen inactivation according to an embodiment of the present disclosure.

FIG. 2 is a schematic diagram of a pathogen control mechanism.

FIG. 3 is a flowchart of a method for manufacturing an electrostatic filter for pathogen inactivation according to an embodiment of the present disclosure.

FIG. 4 is a schematic diagram of an electrostatic filter for pathogen inactivation manufactured according to an embodiment.

FIG. 5 shows the results confirming that the charge retention characteristics are maintained even in a high temperature and high humidity (60° C., RH 90%) environment and the results showing the contact area over time.

FIG. 6 shows a copper foil/graphene contact area.

FIG. 7 shows the pathogen removal effect using the electrostatic filter for pathogen inactivation manufactured according to an embodiment.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed below, but may be implemented in various different forms. Thus, these embodiments are set forth only to make the present disclosure complete, and to entirely inform the scope of the present disclosure to those of ordinary skill in the technical field to which the present disclosure belongs, and the present disclosure is only defined by the scope of the claims.

A shape, a size, a ratio, an angle, a number, etc. disclosed in the drawings for describing the embodiments of the present disclosure are exemplary, and the present disclosure is not limited thereto. The same reference numerals refer to the same elements herein. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list. In interpretation of numerical values, an error or tolerance therein may occur even when there is no explicit description thereof.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

Further, as used herein, when a layer, film, region, plate, or the like may be disposed “on” or “on a top” of another layer, film, region, plate, or the like, the former may directly contact the latter or still another layer, film, region, plate, or the like may be disposed between the former and the latter. As used herein, when a layer, film, region, plate, or the like is directly disposed “on” or “on a top” of another layer, film, region, plate, or the like, the former directly contacts the latter and still another layer, film, region, plate, or the like is not disposed between the former and the latter. Further, as used herein, when a layer, film, region, plate, or the like may be disposed “below” or “under” another layer, film, region, plate, or the like, the former may directly contact the latter or still another layer, film, region, plate, or the like may be disposed between the former and the latter. As used herein, when a layer, film, region, plate, or the like is directly disposed “below” or “under” another layer, film, region, plate, or the like, the former directly contacts the latter and still another layer, film, region, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof.

It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

The features of the various embodiments of the present disclosure may be partially or entirely combined with each other, and may be technically associated with each other or operate with each other. The embodiments may be implemented independently of each other and may be implemented together in an association relationship.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present disclosure relates to an electrostatic filter and film for pathogen inactivation using graphene with improved charge retention ability via a large-area charge injection method, which will be described in a specific manner below.

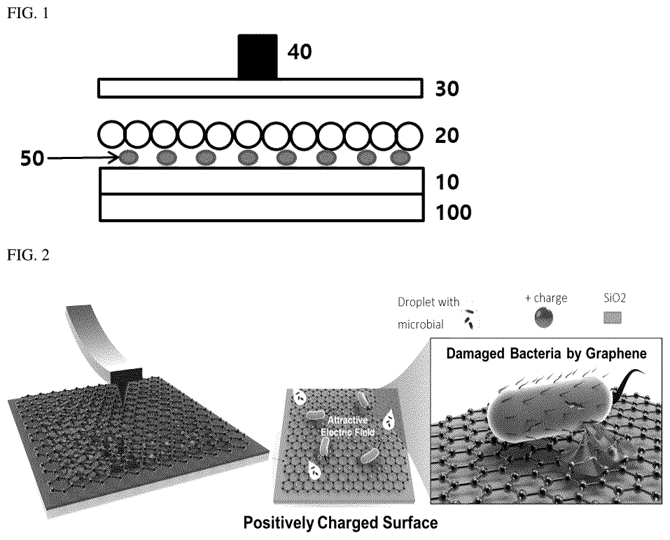

FIG. 1 is a schematic diagram of an electrostatic filter for pathogen inactivation according to an embodiment of the present disclosure.

As shown in FIG. 1, an electrostatic filter and film for pathogen inactivation according to an embodiment of the present disclosure includes a substrate 100; an insulator layer 10 disposed on the substrate 100; and a monolayer graphene layer 20 disposed on the insulator layer 10.

The insulator layer is formed on the substrate (wafer), and the substrate may be omitted in some cases. The insulator layer may include any one of SiO₂, Al₂O₃, and stretchable/flexible polymer (PET, PU, PDMS, PI, etc.).

The graphene layer as a monolayer graphene layer is disposed on the insulator layer.

In the electrostatic filter and film for pathogen inactivation of the present disclosure, in a state in which the graphene layer has been disposed on the insulator layer, a metal foil 30 to which a bias voltage has been applied is placed on an upper surface of the graphene layer, and a force is applied to the foil and then to the graphene layer using a force application means such that the foil and the graphene layer friction-contact each other to generate tribo-electricity, such that electric charges are locally stored between the graphene layer and the insulator layer via a tunneling effect.

In this case, the copper foil may be used as the metal foil, and force may be applied using a weight. The metal foil and the graphene layer friction-contact each other. It is difficult to obtain the tunneling effect with simple friction contact therebetween. A certain amount of the force should be applied thereto to obtain the tunneling effect. Thus, the force is applied thereto using the weight to cause tunneling.

In one example, a bias is applied to the metal foil. The bias may be applied thereto using a DC power supply, etc.

Triboelectric charges are charges generated under triboelectrification between two different materials. Positive and negative charge materials are selected from the triboelectric series. Since the triboelectric charges generated through triboelectrification accumulate on the surface of the friction material, the surface potential of the friction material may be identified using KPFM (Kelvin Probe Force Microscopy) to measure the triboelectric charges.

Tunneling triboelectrification is an effect in which charges generated under friction between two-dimensional materials are located in an insulator under the two-dimensional material through tunneling. For example, when an insulator made of SiO₂ is disposed under the graphene layer, the charges are stored in a space therebetween. Due to this tunneling effect, charges are locally trapped and maintained in the insulator.

In accordance with the present disclosure, the charge injection area of graphene is increased from an existing micro-square meter area to a centimeter square area under the friction between the metal foil and the graphene using the metal foil to which the bias voltage is applied. This may maintain charges over a large area via a single charge injection. When a single charge injection into the graphene is performed at a voltage of 10 V, the tunneling charge may be stored in the distributed manner for 72 hours in a high temperature and humidity (60° C., 90%) environment. The area of the graphene layer may be an area of at least a square centimeter, and may be an area of at least 1 square centimeter, at least 5 square centimeters, at least 6 square centimeters, etc.

Using the electrostatic filter for pathogen inactivation according to one embodiment of the present disclosure, pathogen inactivation may be achieved. Generally, pathogens may include bacteria and viruses, and the surfaces of bacteria and viruses are composed of phospholipids and proteins that have low isoelectric points and carry negative charges. In one example, in the electrostatic filter of the present disclosure, the graphene includes a positive charge. This positive charge inactivates the pathogen when it comes into contact with the pathogen. That is, when the injected charge and the pathogen come into contact with each other, the surface of the pathogen is destroyed, and 99.99% inactivation of the pathogen occurs within 1 minute.

FIG. 2 shows a schematic diagram of a pathogen control mechanism. As shown in FIG. 2, when the pathogen floating in the air comes into contact with graphene, the pathogen's electrons are released therefrom, and the protein thereof is destroyed. Graphene may continuously deactivate the pathogens due to the stored charge therein.

In one example, after using the electrostatic filter of the present disclosure for a certain period of time, the charge may be re-injected into the filter by placing the metal foil to which the bias voltage has been applied on the graphene layer and applying a physical force onto the foil so as to friction-contact the graphene using the weight. Thus, the filter may be reused.

FIG. 3 shows a flowchart of a method for manufacturing an electrostatic filter for pathogen inactivation according to an embodiment of the present disclosure.

As shown in FIG. 3, a method for manufacturing an electrostatic filter for pathogen inactivation according to an embodiment of the present disclosure includes a step of forming an insulator layer on a substrate in S 310; a step of preparing a monolayer graphene layer and transferring the same onto the insulator layer in a wet transfer manner in S 320; and a step of placing a metal foil on the graphene layer, placing a weight on the metal foil, and then applying a voltage to the metal foil to inject charges.

In step S310, the substrate is prepared, and the insulating layer is formed thereon. The substrate may be omitted. The insulating layer may include any one of SiO₂, Al₂O₃, and stretchable/flexible polymer (PET, PU, PDMS, PI, etc.).

In step S320, the monolayer graphene layer is prepared and transferred onto the insulating layer in a wet transfer manner. The step of preparing the monolayer graphene layer and transferring the same onto the insulating layer in a wet transfer manner includes a step of synthesizing a monolayer graphene on the copper foil using a chemical vapor deposition method; a step of spin-coating PMMA (polymethyl methacrylate) on the graphene synthesized on the copper foil; a step of immersing the PMMA-coated graphene into a copper etchant to remove the copper therefrom; and a step of transferring the graphene onto the insulating layer in a wet transfer manner.

In step S330, the metal foil is placed on the graphene layer, the weight is placed on the metal foil, and then the voltage is applied to the metal foil to inject the charge. Any metal foil that exhibits conductivity may be used as the metal foil. For example, the copper foil may be used as the metal foil.

The electrostatic filter for pathogen inactivation according to an embodiment of the present disclosure as manufactured by this method stores and maintains the charges locally in an area between the graphene layer and the insulator layer via the tunneling effect, thereby enabling pathogen inactivation.

Hereinafter, the contents of the present disclosure will be additionally described along with specific examples.

### Example 1

In the example, a 6 cm×6 cm SiO₂ layer was used as the insulator layer, a 6 cm×6 cm copper foil was used as the metal foil, a 5 cm×5 cm monolayer graphene layer was used as the graphene layer, and a 1 kg weight was used as the weight.

The electrostatic filter for pathogen inactivation according to the example was manufactured as follows. The monolayer graphene was synthesized on the copper foil in chemical vapor deposition, and PMMA (polymethyl methacrylate) was spin-coated on the graphene synthesized on the copper foil. The PMMA-coated graphene was immersed in a copper etchant to remove the copper therefrom. The graphene was transferred to the SiO₂ layer (wafer) in a wet transfer manner, and then the PMMA was removed therefrom using acetone. The 6 cm×6 cm copper foil was placed on the transferred 5 cm×5 cm graphene layer, and the 1 kg weight was placed on the upper surface of the copper foil, and 10 V was applied to the copper foil from a DC power supply to inject the charges. The charge retention characteristics of the graphene into which the charges were injected were identified in a high temperature and high humidity (60° C., RH 90%) environment.

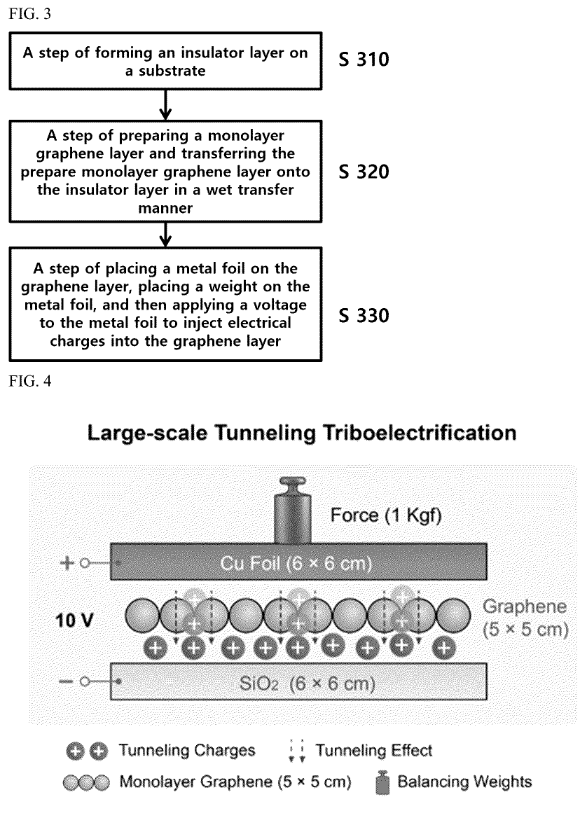

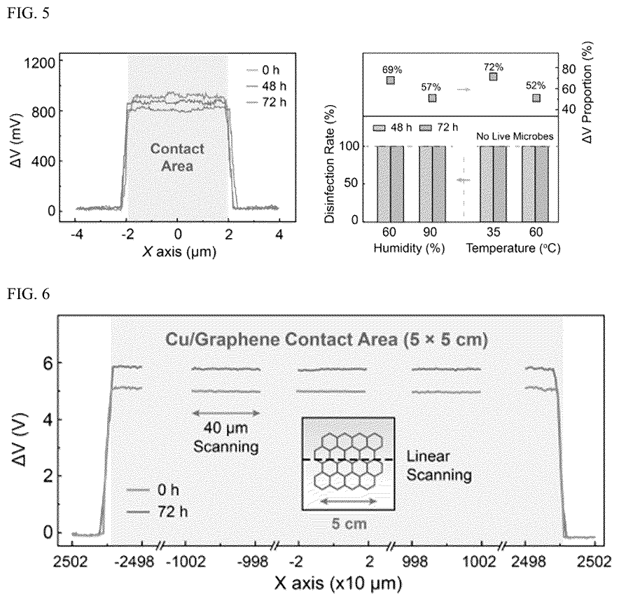

FIG. 4 is a schematic diagram of an electrostatic filter for pathogen inactivation as manufactured by the example. FIG. 5 shows the results indicating that the charge retention characteristics are maintained even in the high temperature and humidity (60° C., RH 90%) environment. FIG. 5 shows the contact area over time. FIG. 6 shows the copper foil/graphene contact area.

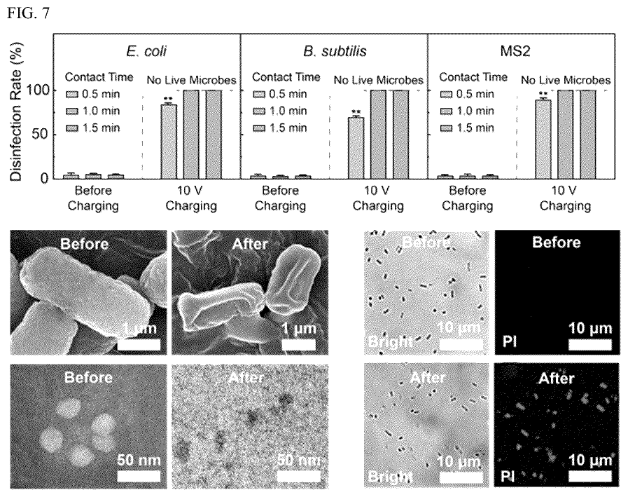

FIG. 7 shows the pathogen killing effect using the electrostatic filter for pathogen inactivation manufactured by the example. (+) charge is injected into graphene, and pathogens with (−) charge are attached thereto. Due to the large-area charge injection, the pathogen removal may be more efficiently achieved than in a conventional manner. The electrostatic filter for pathogen inactivation has stable and strong charge storage ability using the tunneling triboelectrification, and pathogen killing may be effectively achieved in the high temperature and humidity of summer.

The electrostatic filter for pathogen inactivation according to the present disclosure maintains its performance even in a high temperature and humidity environment, increasing the utilization thereof in the pathogen inactivation. Thus, the electrostatic filter for pathogen inactivation according to the present disclosure may be applied to elevator buttons and armrests with a lot of contact with the human hands having the pathogen thereon.

Although some embodiments of the present disclosure have been described above with reference to the accompanying drawings, the present disclosure may not be limited to some embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that some embodiments as described above are not restrictive but illustrative in all respects.

What is claimed is:

1. An electrostatic filter and film for pathogen inactivation, comprising:
an insulating layer disposed on a substrate; and
a monolayer graphene layer disposed on the insulating layer,
wherein a metal foil is placed on an upper surface of the graphene layer, and the metal foil is pressed against the graphene layer under a physical force so as to cause friction therebetween while a bias voltage is applied to the metal foil, such that electric charges are locally stored in an area between the graphene layer and the insulating layer under a tunneling effect.

2. The electrostatic filter and film for pathogen inactivation of claim 1, wherein the insulating layer includes one selected from SiO₂, Al₂O₃, and stretchable/flexible polymer.

3. The electrostatic filter and film for pathogen inactivation of claim 1, wherein the graphene layer has an area of 1 centimeter square or larger.

4. The electrostatic filter and film for pathogen inactivation of claim 1, wherein the metal foil is a copper foil, wherein the physical force is applied using a weight.

5. The electrostatic filter and film for pathogen inactivation of claim 1, wherein the charge injected into the graphene layer is stored for 72 hours at a temperature of 60° C. and a humidity of 90%.

6. The electrostatic filter and film for pathogen inactivation of claim 1, wherein after using the electrostatic filter and film for a predefined period of time, the metal foil is placed on the upper surface of the graphene layer, and the metal foil is pressed against the graphene layer under a physical force so as to cause friction therebetween while the bias voltage is applied to the metal foil, such that new electric charges are re-stored in the area between the graphene layer and the insulating layer under a tunneling effect, and thus the electrostatic filter and film is reusable.

7. A method for manufacturing an electrostatic filter and film for pathogen inactivation, the method comprising:
a step of forming an insulator layer on a substrate;
a step of preparing a monolayer graphene layer and transferring the prepare monolayer graphene layer onto the insulator layer in a wet transfer manner; and
a step of placing a metal foil on the graphene layer, placing a weight on the metal foil, and then applying a voltage to the metal foil to inject electrical charges into the graphene layer, such that electric charges are locally stored in an area between the graphene layer and the insulating layer under a tunneling effect.

8. The method for manufacturing the electrostatic filter and film for pathogen inactivation of claim 7, wherein the step of preparing the monolayer graphene layer and transferring the prepare monolayer graphene layer onto the insulator layer in the wet transfer manner includes:
a step of synthesizing a monolayer graphene on a copper foil using a chemical vapor deposition;
a step of spin-coating PMMA (polymethyl methacrylate) on the graphene synthesized on the copper foil;
a step of immersing the PMMA-coated graphene into a copper etchant to remove copper therefrom; and
a step of transferring the graphene onto the insulating layer in a wet transfer manner.

9. The method for manufacturing the electrostatic filter and film for pathogen inactivation of claim 7, wherein the insulating layer includes one selected from SiO₂, Al₂O₃, and stretchable/flexible polymer.

10. The method for manufacturing the electrostatic filter and film for pathogen inactivation of claim 7, wherein the graphene layer has an area of 1 centimeter square or larger.

11. An electrostatic filter and film for pathogen inactivation manufactured according to the method of claim 7.
